(19) United States
(12) Patent Application Publication
(10) Pub. No.: US 2026/0295260 A1
(43) Pub. Date: Oct. 1, 2026

(54) SLEEP STAGE DETECTION AND THERAPY ADJUSTMENT

(71) Applicant: THE ALFRED E MANN FOUNDATION FOR SCIENTIFIC RESEARCH, VALENCIA, CA (US)

(72) Inventor: BRIAN SHELTON, VENTURA, CA (US)

(73) Assignee: THE ALFRED E MANN FOUNDATION FOR SCIENTIFIC RESEARCH, VALENCIA, CA (US)

(21) Appl. No.: 19/247,138

(22) Filed: Jun. 24, 2025

Related U.S. Application Data
(60) Provisional application No. 63/777,903, filed on Mar. 26, 2025.

(51) Int. Cl. A61N 1/36

(52) U.S. Cl. CPC A61N 1/3611; A61N 1/36132; A61N 1/36139

(57) ABSTRACT

The present disclosure generally relates to implantable stimulation systems and methods for tailoring stimulation to the sleep stage of a subject. The disclosed methods may relate to monitoring sleep stages, detecting respiratory events (e.g., apneas and/or hypopneas), and generating sleep quality metrics for individual sleep stages that are, in turn, used to titrate optimal stimulation parameters for each sleep stage. Such titrations may be beneficial for conserving system energy, increasing therapeutic efficacy, or improving patient comfort. In certain implementations, the systems and methods described could be used stimulate a hypoglossal nerve (HGN) to treat a breathing disorder such as obstructive sleep apnea (OSA).

### CROSS-REFERENCE TO RELATED APPLICATIONS

This application relates to and claims priority from U.S. provisional patent application No. 63/777,903, filed on Mar. 26, 2025, and entitled “Sleep Stage Detection and Therapy Adjustment”.

### FIELD

The present disclosure generally relates to implantable stimulation systems and methods for adjusting stimulation parameters based on an individual's sleep stage.

### BACKGROUND

A variety of implantable medical devices (IMDs) have been developed to provide health-related therapies and monitoring. Notable examples include pacemakers, cardioverter-defibrillators, cardiomyostimulators, neural stimulators, drug infusion pumps, and various physiological sensors. This disclosure provides a system and methods in the context of implantable nerve stimulators that are optionally configured for treating Obstructive Sleep Apnea (OSA), although the underlying principles could apply to a variety of different IMDs and/or other indications, as well.

Obstructive Sleep Apnea (OSA) is a prevalent sleep disorder characterized by repeated episodes of partial or complete obstruction of the upper airway during sleep, leading to disrupted sleep patterns and reduced oxygen saturation levels in the blood. This condition can result in significant adverse health effects, including cardiovascular disease, daytime fatigue, and cognitive impairments. Traditional treatment methods, such as continuous positive airway pressure (CPAP) therapy, often suffer from low patient compliance due to discomfort and inconvenience, prompting the development of alternative therapeutic approaches.

Implantable stimulators, including implantable pulse generators (IPGs), have become a promising solution for managing Obstructive Sleep Apnea (OSA). These devices deliver electrical pulses to targeted nerves or muscles—such as the hypoglossal nerve (HGN) or genioglossus muscle—to help maintain airway patency during sleep, reducing the frequency and severity of apneic events.

A significant challenge associated with implantable stimulation systems is their present inability to tailor stimulation for different sleep stages. Sleep is a complex process consisting of various phases of non-rapid eye movement (NREM) and rapid eye movement (REM) sleep, each defined by distinct physiological states. The effectiveness and necessity of stimulation can vary across these stages, and a patient's tolerance to stimulation is also influenced by their sleep stage. For instance, muscle tone naturally decreases during REM sleep, potentially necessitating a different stimulation approach compared to NREM sleep, and a stimulation intensity that is tolerable in one sleep stage can sometimes cause discomfort in another. The energy efficiency of a system is also compromised when stimulation is not tailored to the sleep stage, as continuous or inappropriate stimulation consumes more power, resulting in more frequent battery replacements or recharging.

Many conventional systems are programmed to perform a titration to determine stimulation parameters (including, e.g., a pulse width, a pulse amplitude, a pulse frequency, and a duty cycle) that elicit a desirable physiological response. However, most conventional systems apply the same parameters for each instance of stimulation, regardless of the patient's sleep stage. This results in excessive or insufficient stimulation being delivered at differing points in the sleep cycle and may cause discomfort, such as throat soreness, if a relatively aggressive stimulation protocol is applied during lighter stage of sleep.

Thus, there is a need for advancement in the technology surrounding implantable stimulation systems, particularly with regard to their present inability to adapt stimulation to different sleep stages. Such improvements could enhance therapeutic outcomes, prolong system battery life, minimize side effects, and improve patient compliance, ultimately providing a more effective and patient-friendly solution.

### SUMMARY

The following presents a simplified summary of one or more aspects of the present disclosure to provide a basic understanding. Any or all of the discussed aspects/features could be interchangeably applied between examples. Unless otherwise stated, no single aspect or feature is essential to achieve the technical effects/solutions discussed.

In certain aspects, the present disclosure provides a stimulation system that could optionally be used to stimulate the hypoglossal nerve to treat Obstructive Sleep Apnea (OSA).

In some examples, the system includes an implantable stimulator, such as an IPG, a controller having a processor and a memory, and at least one sensor for collecting physiological data. The controller processes the physiological data to determine sleep stage, detect respiratory events, and generate sleep quality metrics (SQMs) for individual sleep stages. During a titration, the controller is configured to compute successive SQMs for a particular sleep stage while systematically adjusting a stimulation protocol. In exemplary implementations, such a titration could be used to fine-tune respective sets of stimulation parameters for each of a plurality of sleep stages experienced by the subject, to improve the efficiency and comfortability of stimulation applied throughout the night. Systems in accordance with this disclosure may advantageously display improved power efficiency (e.g., components of the OSA stimulation system may be disabled or switched to a low-power mode when a subject is found to be awake or in a sleep stage where stimulation is reduced or unnecessary), accuracy, and/or functionality compared to current systems. Various other features and advantages will become clear in later portions of this disclosure.

In some examples, physiological data collected by the system may relate to or include: cardiac data, motion data, respiratory data, temperature readings, blood oxygen levels, muscle activity, heart rate, body movement, and/or body position. This data can be used to identify sleep disturbances (e.g., apneas, hypopneas, and/or wake events) and assessments of heart rate variability (HRV) or respiratory rate variability (RRV) may be used to differentiate sleep stages, in certain implementations.

In some examples, the at least one sensor may include an external sensor, or a combination of implantable and external sensors. In idealized embodiments, the system includes at least one implantable sensor.

In some examples, the at least one sensor includes a first sensor unit for detecting biomarkers indicative of respiratory effort. The first sensor unit may include at least one of: an inertial measurement unit (IMU, e.g., an accelerometer or gyroscope), a microphone, a pressure sensor, an electrocardiogram (ECG) sensor, or an electroneurography sensor (ENG). It is expressly contemplated that any of the systems described herein could comprise a plurality of sensors, each positioned independently on or in proximity to the patient, and such systems may utilize physiological data from any or all sensors when determining sleep stage.

In some examples, the at least one sensor further includes a second sensor unit for detecting biomarkers indicative of a heart rate of the subject. The controller is configured to receive one or more biomarkers from the second sensor unit to determine a heart rate of the subject, determine changes in heart rate of the subject over a period of time, and determine a level of wakefulness and/or a sleep stage of the subject based on, e.g., a change in heart rate of the subject over a period of time. The second sensor unit may include at least one of: an IMU, a pressure sensor, a photoplethysmography (PPG) sensor, an ECG sensor, a triaxial accelerometer, and/or gyroscope. In some examples, the second sensor unit is optionally configured to detect both respiration and heart rate of a subject.

In another aspect, the present disclosure provides a system that is capable of adjusting the intensity of electrical stimulation applied by an implantable pulse generator (IPG), depending on the sleep stage of a subject. A controller processes physiological data collected by at least one sensor to determine sleep stage, detect respiratory activity/events (such as, e.g., apneas and/or hypopneas), and computes sleep quality metrics (SQMs) for individual sleep stages based on the number of respiratory events detected in each stage. During a titration, an adjustment is made to a stimulation protocol associated with a particular sleep stage. Before and after each adjustment, the controller computes an SQM, and the SQMs are compared to assess whether sleep quality has improved. Also, at least one patient input is collected and transmitted to the controller during the titration, and in certain examples the patient input relates to a perceived comfortability and/or satisfaction with stimulation. Using the SQMs and the patient input, the controller can algorithmically determine, for each sleep stage, a stimulation intensity that maximally improves sleep quality without causing discomfort.

In various examples, the controller which executes the computational processes associated with the titration could be implanted or external. For instance, the titration could be performed a controller housed within the IPG, or a controller associated with an external device could be used to offload computational tasks from the IPG.

In another aspect, the present disclosure provides a method for titrating (i.e. adjusting) one or more stimulation protocols associated with respective sleep stages. The method involves monitoring sleep stages and logging the quantity of respiratory events that occur within each sleep stage. In particular, at least one sensor is used to collect physiological data over successive first and second periods. Using such data, first and second sleep quality metrics (SQMs) are generated based on respective quantities of respiratory events that are detected during the first and second periods. Next, the first and second SQMs are compared, a patient input is received, and the controller determines if the value of one or more stimulation parameters associated with the given sleep stage should be adjusted (i.e. increased or decreased).

In another aspect, a system is provided for selecting a stimulation protocol from a plurality of stimulation protocols in response to detecting a transition in the sleep stage of a subject. The plurality of stimulation protocols includes at least a first stimulation protocol and a second stimulation protocol associated with respective sleep stages. The first stimulation protocol is selected when the subject is found to be in a first sleep stage, and the second stimulation protocol is selected and when the subject is found to be in a second sleep stage.

In various examples, the stimulation system could be configured to apply closed-loop or open-loop stimulation. In closed-loop embodiments, the system could be configured to deliver stimulation in response to detecting any of: an apnea or hypopnea, a particular phase of respiration, or a heart rate that falls above or below a predefined threshold. Alternatively, open-loop stimulation could be applied according to a predefined schedule or timing.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a schematic drawing of a stimulation system in accordance with some examples.

FIG. 2 is a graph of a stimulation protocol which includes pulse amplitudes that are varied between sleep stages.

FIG. 3 is a flow diagram of a titration algorithm for optimizing a parameter of a stimulation protocol associated with a particular sleep stage.

FIGS. 4A-4C provide examples of prompts for a patient input displayed on an external remote.

FIG. 5 is a flow diagram illustrating a method for selecting a stimulation protocol based on detected change in sleep stage.

FIG. 6 is a flow diagram illustrating a method for adjusting (or “titrating”) a stimulation intensity for each sleep stage.

### DETAILED DESCRIPTION

The present disclosure provides, in part, a system and methods for controlling at least one parameter of an electrical pulse that is applied by an implantable stimulation system, depending on the sleep stage of a subject. The system can be optionally adapted for treating OSA and includes at least one sensor for obtaining physiological data (e.g. cardio-respiratory data and/or motion data) that is processed to determine sleep stage, detect respiratory events, and to compute sleep quality metrics (SQMs). Such data can, in turn, be used to titrate optimized stimulation parameters for each sleep stage, resulting in improved power efficiency, treatment efficacy, and patient comfort, compared to some conventional OSA stimulation systems.

Along with stress, breathing disorders are the most common cause of compromised sleep, and among sleep related breathing disorders, sleep apnea is the most common. When a person is suspected of having a sleep related problem including sleep related breathing disorders (SBDs) they typically undergo a polysomnogram (PSG) study in a sleep lab. PSG studies measure eight key parameters during a course of a night's sleep: airflow, breathing rate and effort, blood oxygen level, body position, eye movement, muscle electrical activity, heart rate, and brain activity. Among these parameters, brain activity is measured using EEG which can accurately determine the amount of time spent in each of the 4 sleep stages (light sleep or “N1”, intermediate sleep or “N2”, deep sleep or “N3”, and REM sleep). This EEG data coupled with wakefulness (a parameter based on the number and duration of awakenings) provides the most accurate assessment of sleep quality. However, clinical EEG systems use twenty-five or more electrodes distributed on the scalp, precisely located and often affixed with a viscous conductive material by highly trained personnel, to perform their measurements. As such, EEG systems are not conducive to routine use at home, not only because EEG studies are difficult to administer, but also because most people would not have difficulty sleeping in view of the complex wiring and equipment required by EEG systems.

In the last decade or so, a plethora of activity trackers, fitness trackers, and sleep trackers have been developed which are typically wearable devices (e.g., wrist-worn devices) that use sensors to detect actigraphy, heart rate, respiration rate, and/or blood oxygen levels and also impute physiological biomarkers like heart rate variability (HRV), body position, number of minutes active, number of steps taken, respiration rate, and even time spent in various sleep stages and hence a sleep score indicative of the quality of sleep. Recent estimates of the accuracy of the imputed time spent in each sleep stage vary between 50 and 80%. Accordingly, there is a need for more accurate sleep stage tracking. Systems according to the present disclosure may be used, among other things, to improve the accuracy of sleep stage determinations, particularly in individuals that happen to have an implantable device (e.g., an OSA stimulation system).

Although the present disclosure is not strictly limited to any one method for determining sleep stage, suitable methods might involve collecting cardiac or respiratory data (using, e.g., an implanted accelerometer, gyroscope, or EcG) for tracking heart rate variability (HRV) or respiratory rate variability (RRV). A combined assessment of HRV and RRV could also be used to increase the accuracy of the system, and a variety of additional sensors could be provided to supplement the dataset and improve the accuracy of sleep tracking over conventional products.

By way of example and not limitation, examples of suitable sensors/methods for determining sleep stage are described within U.S. Pat. No. 12,1786,00, entitled “Wakefulness and Sleep Stage Detection Using Respiration Effort Variability”, issued Dec. 31, 2024, and assigned to the present Applicant. Systems of the present disclosure could employ any of the sensors/methods described to determine sleep stage.

The systems described below are capable of generating sleep quality metrics (SQMs) based on sensed data. An SQM, as described in the context of this disclosure, provides a numeric assessment of sleep quality in relation to a particular sleep stage. For example, an SQM could be determined based on, e.g., the total number of adverse respiratory events (e.g. apneas or hypopneas) detected to have occurred while the subject was in a particular sleep stage (e.g. while in light sleep), using physiological data collected over a period of one or more nights. In different examples, data used for determining/calculating sleep quality metrics could include any one or a combination of cardiac data, respiratory data, or patient motion data.

The technical process of classifying respiratory activity to differentiate apnea/hypopnea events from regular breathing typically involves the use of a trained classifier to interpret the sensed data. The term “classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), deep learning systems, and random forest classifiers. Systems of the present disclosure are intended to be modular, may include a variety of sensors and/or different sensor combinations, and thus different algorithms or classification schemes could be used in different embodiments to differentiate apnea/hypopnea events from regular breathing. For instance, U.S. published application No. 2024/0138705A1, which is entitled “Systems and Methods for Detecting Apneas and Hypopneas”, published May 2, 2024, provides at least one suitable example.

The ability to generate SQMs for individual sleep stages is leveraged by systems of the present disclosure during a titration. Through the titration, a respective set of stimulation parameters can be optimized for each sleep stage, to enhance the therapeutic efficacy and comfortability of stimulation applied in each stage. For instance, if one or more SQMs computed by the system indicate that a subject has experienced relatively poor sleep quality during a particular sleep stage, then the value of one or more stimulation parameters associated with that sleep stage may be systematically adjusted during a period of titration. Throughout the titration, great care is taken to ensure that stimulation is never permanently adjusted to a level that causes discomfort, and the subject may be periodically prompted to provide an input relating to their perceived level of comfort or satisfaction with therapy. If the patient input suggests that a therapy session caused discomfort, then the system may automatically reduce the intensity of stimulation applied during future sessions. Following each adjustment, a new SQM is computed and compared against the historical SQM(s) to assess the degree of sleep quality improvement within the given stage.

### I. Definitions

As used herein, the term “stimulation protocol” denotes a set of predefined therapeutic stimulation parameters (such as pulse amplitude, frequency, pulse width, and duty cycle) that is applied during one or more sleep stages.

As used herein, the term “titration” refers to a process of methodically adjusting a stimulation protocol over a plurality of therapy sessions to optimize its therapeutic efficacy, to maximize patient sleep quality, and/or to minimize patient discomfort.

As used herein, the terms “apnea” and “hypopnea” are to be understood with reference to the current American Academy of Sleep Medicine (“AASM”) definitions for these terms. The AASM guidelines define a “hypopnea” (in an adult patient) as a ≥30% reduction in nasal pressure, nasal airflow, or some other hypopnea sensor that last for ≥10 seconds and that corresponds to an oxygen desaturation event. A hypopnea oxygen desaturation event can either be defined as a ≥3% drop from the pre-event baseline with an arousal or as a ≥4% drop from the pre-event baseline without consideration of an arousal. The AASM guidelines further define an “apnea” as a cessation of airflow for ≥10 seconds.

As used herein, the term “controller” relates to an element including one or more processors. Such controllers may include other components as well, such as a memory and a wireless communication unit (e.g., a Bluetooth transceiver).

As used herein, the term “Apnea-Hypopnea Index” (“AHI”) refers to a metric calculated by dividing the total number of apneas and hypopneas in each sleep stage by the subject's total sleep time in said stage. It is understood that the AHI is an exemplary metric that may be used by the systems described herein. In some aspects, alternative metrics such as the “Respiratory Disturbance Index” (“RDI”), “Respiratory Event Index” (“REI”), or “Oxygen Desaturation Index” (“ODI”) may be used. The RDI is defined as the total number of apneas, hypopneas and Respiratory Effort-Related Arousals (“RERAs”), divided by the subject's total sleep time in a given sleep stage. The REI is defined as the total number of apneas and hypopneas, divided by the total monitoring time in hours. An AHI or REI <5 per hour is normal (for adults); an AHI or REI of 5-14.9 per hour is indicative of mild OSA; an AHI or REI of 15-29.9 per hour is indicative of moderate OSA; and an AHI or REI of ≥30 per hour is indicative of severe OSA. “ODI” refers to a metric to quantify the number of times per hour that a person's blood oxygen level drops by a certain degree from baseline during sleep, and the American Academy of Sleep Medicine (AASM) defines the ODI as the number of times per hour of sleep that the blood oxygen level drops by at least 3% from baseline.

As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It will be understood that when an element or layer is referred to as being “on”, “connected to”, “coupled to”, or “adjacent to” another element or layer, it can be directly on, connected to, coupled to, or adjacent to the other element or layer, or one or more intervening element(s) or layer(s) may be present. In contrast, when an element or layer is referred to as being “directly on,” “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Although various aspects of the present disclosure are shown and described in exemplary context implanted medical devices (IMDs), applications of the present invention are not so limited and could, for example, be applied to a wide variety of therapeutic stimulation devices, be they fully implantable stimulation, partially implantable, or entirely external. The scope of this disclosure is intended to encompass all known stimulation modalities including, for example, electrical stimulation, light stimulation (e.g. photo biomodulation or low-level laser therapy), vibratory stimulation, chemical stimulation, and thermal stimulation, among others.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the application and design constraints imposed on the overall system.

By way of example, an element, or any portion of an element, or any combination of elements may be implemented as a “processing system” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, computer-readable media can comprise a random-access memory (“RAM”), a read-only memory (ROM), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

### II. Drawings and Example Embodiments

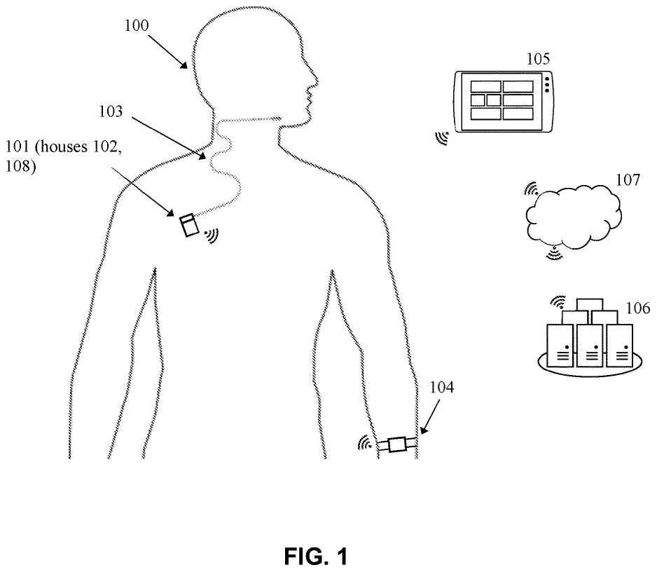

FIG. 1 is a diagram illustrating an example embodiment of a stimulation system 100 (or “system” 100) configured for controlling a parameter of a pulse applied by a medical device 101, which in this instance comprises an implantable pulse generator (IPG) 101. IPG 101 is implanted in the chest region of a subject and is coupled to a stimulation lead 103. A distal end of the stimulation lead 103 is situated in the neck of the patient in the vicinity of the hypoglossal nerve (HGN) to direct pulses of electrical stimulation from IPG 101 to the HGN. Although not clearly shown in the figures, stimulation lead 103 can include a plurality of electrodes coupled to its distal end. Such electrodes may optionally function as anodes or cathodes for stimulating the HGN and/or for sensing electrical activity in the vicinity of the HGN, and, in certain embodiments, the electrodes may be carried by the body of a biocompatible neural interface such as, e.g., a nerve cuff, a paddle lead or electrode array, or a helical cuff.

A controller 102 is housed within IPG 101 and communicates with one or more sensors 108, 104. The sensors 108 and 104 are used for sensing physiological data, which could include any of respiratory data, cardiac data, and/or motion data. The physiological data is received and interpreted by controller 102 to determine sleep stage and track respiratory events (e.g. apneas and/or hypopneas). An external controller 105 and optional cloud-based components 106, 107 are also illustrated.

The implanted controller 102 is configured to detect/classify medically relevant respiratory events (e.g. to detect apnea or hypopnea events) using a trained classifier executed by the implanted controller 102 to interpret sensed data. The classifier may also, in some examples, be configured to differentiate inspiratory and expiratory phases of patient respiration. Such classifications could alternatively be performed by external controller 105 or by a cloud-based server in different embodiments (e.g., it may be advantageous to offload the computation required for a classification to an external device, rather than using the power and limited processing capabilities of an implanted controller 102). In various embodiments, the trained classifier can analyze sensor data collected from any number of implanted or external sensors (108, 104) and could utilize any classification algorithm suitable for the given sensors. In various embodiments, either of sensors 108 and/or 104 could comprise a sensor unit that includes a plurality of sensors.

Some systems 100 the present disclosure may utilize one or more implanted sensors 108 (such an implanted inertial measurement unit (IMU) for tracking patient motion, respirations, and/or heartbeats) without the need for data collected from any external sensors 104. However, in some cases the implanted system may not have all the sensors 108 needed to measure each of the parameters described. Thus, external sensors 104 may be provided to supplement the dataset, to, for instance, include actigraphy, heart rate, blood oxygen level, blood pressure, EEG, single or low channel EEG, in-ear EEG, other brain activity like FNIRS (functional near infra-red spectroscopy), EMG, eye movement (EOG), and environmental data such a temperature, humidity, extraneous noise, etc. It is understood that systems 100 of the present disclosure are modular in that they may take into account sensor data collected using any combination of implanted and external sensors (108 and 104, respectively).

In this example, external sensor 104 is a wrist-worn sensor integrated into a smart watch or fitness tracker (e.g., a heart rate sensor, inertial sensor, SpO2 sensor, etc.). Implanted sensor 108 is an IMU (e.g. an accelerometer and/or gyroscope) configured for tracking patient chest motion associated with respiration, gross patient motion, and could also be used to detect patient heartbeats in certain implementations. The controller 102 is configured to handle signal processing, data storage, and operation of system 100. Implanted controller 102 may also include a communication unit (e.g. a transceiver) to allow for wireless communication between the IPG 101 and a user application 109 executed on external controller 105. In this example, implanted controller 102 can communicate with each of internal sensor 108, external sensor 104, and external controller 105.

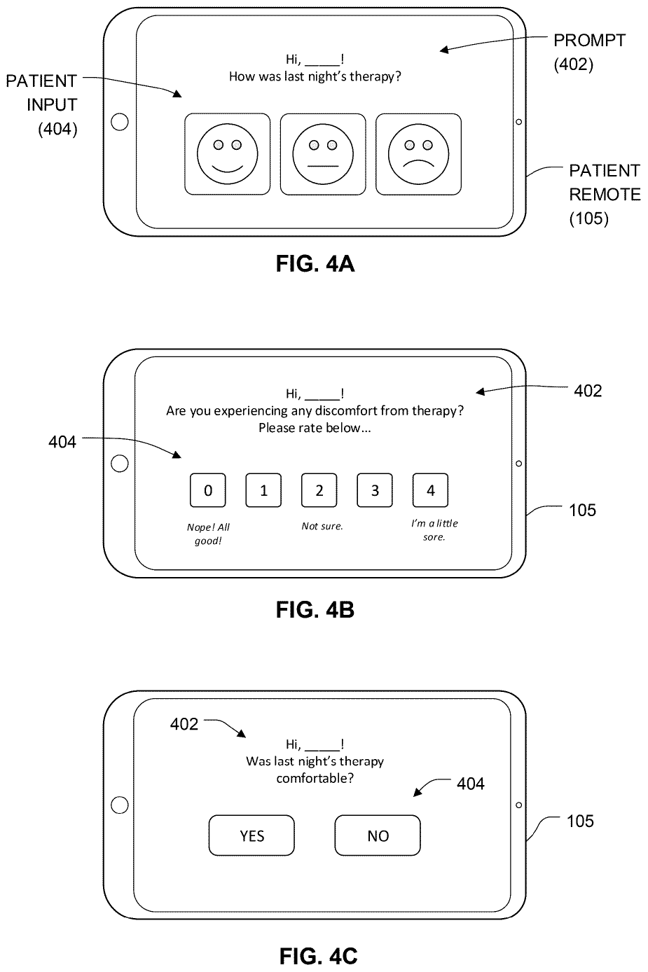

External controller 105 may be, e.g., a dedicated controller, such as a clinician programmer or patient remote, with a text-based or graphical user interface, or software executed on a user's smart phone, tablet, computer, or other multi-purpose electronic device. The implanted controller 102 and/or the external controller 105 may be configured to communicate with one or more local, remote, or cloud-based servers. In this case the external controller 105 can communicate with a remote server 106 via intermediary cloud-based infrastructure 107. External controller 105 may be configured to execute a user application configured to communicate with a clinical application via the intervening cloud infrastructure 107, allowing a remote clinician or patient to interact with the external controller 105 or the implanted controller 102. This configuration may allow for a clinician or patient to view a sleep quality metric, to view sensor data, to view and/or modify one or more settings of the OSA stimulation system 100, to initiate or terminate a titration, or to allow a patient to provide an input related to their perceived comfort level or satisfaction with therapy, as will be later described in reference to FIGS. 4A-4C. As another example, a clinician may be able to edit one or more parameters of a stimulation protocol stored on the external controller 105, which may in turn be transmitted to the implanted controller 102 to change how sleep stage-specific stimulation parameters are applied by the IPG 101 during therapy.

System 100 may simplify and improve upon existing designs for commercial OSA stimulation systems by incorporating additional sensor functionality into one or more implanted components of the system. For example, system 100 may include an IMU in addition to one or more implanted sensors 108, such as a combination of ECG sensors, SpO2 sensors, and/or a microphone, to classify apnea and hypopnea events, monitor patient sleep stage, and compute SQMs for respective sleep stages. Depending on the embodiment, such SQMs could comprise a sleep quality metric such as an AHI, and AHI-like metric, an ODI, or an ODI-like metric for each sleep stage.

The SQMs are, in turn, used to auto-titrate one or more parameters (e.g., pulse amplitude, pulse width, or duty cycle) of a stimulation protocol that is applied by IPG 101 during a particular sleep stage. Such SQMs might include daily sleep quality metrics (“D-SQMs”) and/or average sleep quality metrics (“A-SQMs”). D-SQMs are determined daily, based on the number of apnea or hypopnea events detected in a given sleep stage using data collected over one night, whereas average sleep quality metrics (A-SQMs) are determined periodically based on the average number of apneas or hypopneas experienced by a subject while in a given sleep stage, based on data collected over a plurality of nights. A-SQMs are therefore less likely to be affected by day-to-day fluctuations in sleep quality that might occur due to factors other than an adjustment to therapy. Thus, when performing a titration, it is often preferable for system 100 to compute and compare A-SQMs to more clearly accurately assess how a therapy adjustment has affected a patient's sleep.

Titrations in accordance with the present disclosure may be periodically performed over a predetermined period (e.g. over a period of 7 days), may be repeated on a scheduled basis (e.g. every other month), or could be initiated by system 100 in response to a command provided by a user or clinician on external controller 105. During the titration, system 100 monitors the frequency of apneic events in each sleep stage over a predetermined period of one or more days. At the end of the period, either controller (102 or 105) may be used to compute an SQM for any one sleep stage for comparison against one or more historical SQMs stored in the system memory (i.e. an SQM that was computed during a prior titration). Depending on the assessment, system 100 may then incrementally adjust (i.e. increase or decrease) the value of one or more stimulation parameters, collect physiological data over another period, and compute another SQM to assess any changes in sleep quality. This process may be repeated cyclically until sleep quality ceases to improve with successive adjustments.

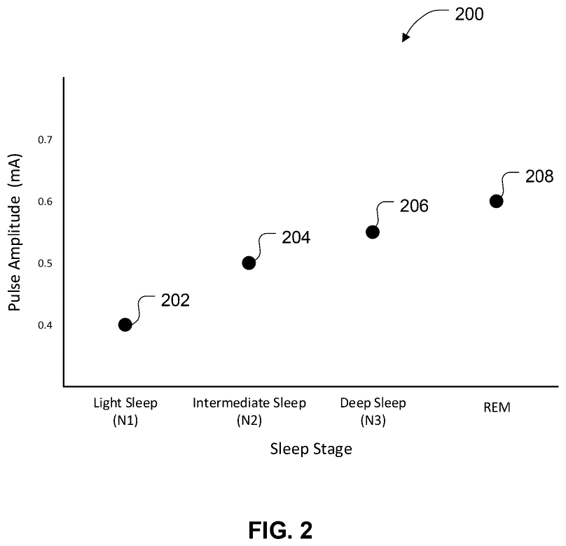

FIG. 2 is a chart 200 depicting pulse amplitudes 202, 204, 206, 208 that are varied between light sleep (N1), intermediate sleep (N2), deep sleep (N3), and REM sleep. In this particular example, amplitudes 202, 204, 206, and 208 are ramped, increasing in intensity as the patient progresses into deeper stages of sleep. Each amplitude (202, 206, 206 and 208) is set at a value corresponding with a maximum-effective pulse intensity for the given sleep stage, as determined by the titration. Each value is saved within a respective stimulation protocol stored in the system memory so that, during future therapy sessions and whenever stimulation is to be applied, controller 102 can detect the subject's present sleep stage and instruct IPG 101 to apply the stimulation with the appropriate intensity.

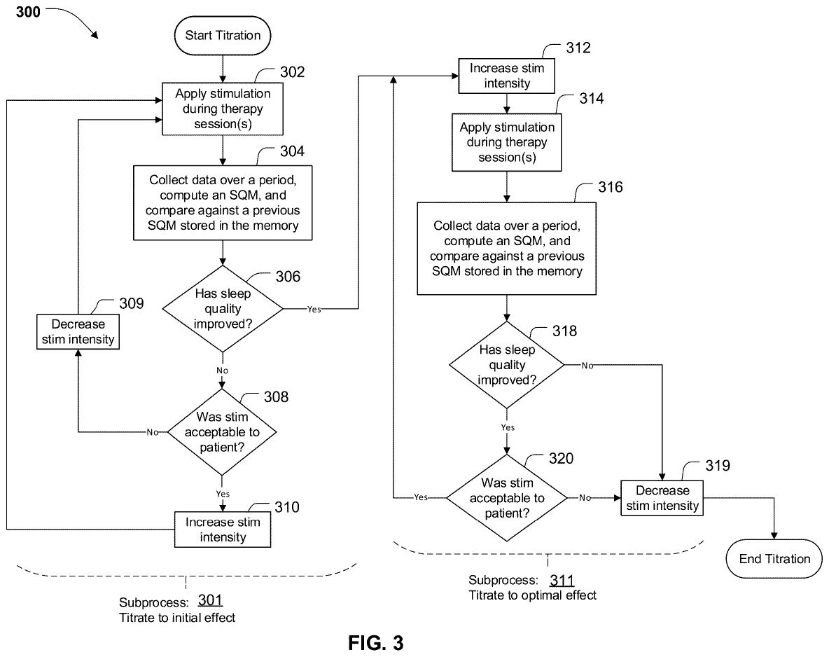

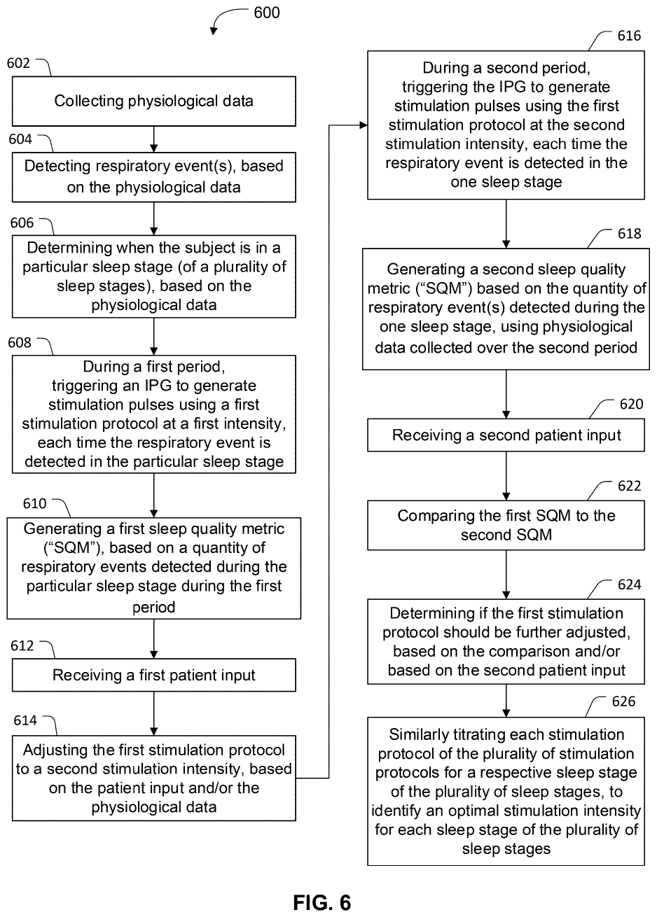

In different examples, the particular values of pulse amplitudes 202, 204, 206, and 208 are likely to differ from those shown, as each patient can have different physiological tolerances/receptiveness to stimulation, so titrations of the present disclosure may not always produce pulse amplitude values that are ramped in the manner shown. Further, although only one parameter-pulse amplitude-is shown to vary between sleep stages, the exemplary titration methods 300 and 600 described below and with reference to FIGS. 3 and 6 could be used to optimize a different parameter or multiple parameters for each sleep stage. For instance, alternate embodiments might include differing pulse frequencies, pulse widths, pulse shapes, and/or duty cycles.

FIG. 3 provides a flowchart illustrating steps associated with an exemplary titration (or “titration algorithm”) 300. Titration 300 is useful for determining an optimal stimulation intensity for each sleep stage. Because many patients have differing sensitivities to stimulation depending on their sleep stage, it is possible that additional therapeutic benefit could be gained by applying higher or lower intensity stimulation in various stages. At the conclusion of titration 300, the identified stimulation intensity value should ideally correspond with the minimum value that provides the highest attainable improvement in sleep quality without causing discomfort. Titration 300 may be implemented by any processor in communication with the system 100 and uses collected physiological data and patient inputs (on external controller 105) to influence how the stimulation protocol is adjusted over time. As noted above, such patient inputs typically comprise an assessment of the subject's perceived comfort level or satisfaction with therapy, and a prompt requesting a patient input may be presented to the patient in the morning following a therapy adjustment so that, in the event that the adjustment caused discomfort, algorithm 300 can to take corrective measures to reduce the intensity of stimulation applied during future therapy sessions.

Algorithm 300 includes two subprocesses. A first subprocess 301 (which includes steps 302, 304, 306, 308, 309 and 310) is conducted to initially find a value of the target parameter (in this case, a pulse intensity value) that, when applied during the given sleep stage, causes an initial improvement in sleep quality. Second subprocess 311 (which includes steps 312, 314, 316, 318, 319, and 320) is conducted after subprocess 301 to fine-tune the target parameter value for optimal therapeutic effects.

Titration algorithm 300 begins with conducting a first therapy session 302 in which pulses of electrical stimulation are applied to a hypoglossal nerve of the patient. During the first therapy session and when the patient is determined to be in the particular sleep stage, IPG 101 generates pulses of electrical stimulation at an initial pulse intensity (or amplitude). The default intensity value may be encoded into the system memory, could have been programmed by a surgeon during a fitting/implantation procedure, or may have been established during a previous titration. During each therapy session, data is collected (using, e.g., sensors 108 and/or 104) to monitor patient respiratory events occurring within the particular sleep stage. Using this data, one or more SQMs are computed 304, stored in the system memory, and compared 306 to determine if sleep quality in the given sleep stage has recently changed, due to the adjustment to the stimulation protocol or otherwise.

In subprocess 311, stimulation intensity is initially increased in step 312, and the intensity can be further increased with each cyclic iteration of subprocess 311 to explore whether it's possible to further improve sleep quality (within the particular sleep stage of interest) through fine-tuned adjustments.

With each cyclic iteration of step 312, stimulation intensity could be increased in a consistent or variable manner, depending on the embodiment. For example, each cycle of step 312 might involve a predefined value or percentage increase (such as a +0.01 mA or a +5% increase in stim intensity). Alternatively, the degree/amount of adjustment provided in step 312 could become progressively smaller with each cycle of subprocess 311, as this can provide the system with added granularity for determining an optimal pulse intensity value while avoiding overstimulation. As noted above, if any such adjustments are indicated by the patient (e.g. in step 320) to have caused discomfort, the system can employ corrective measures to reduce the stimulation intensity (e.g. in step 319) and/or to revert the stimulation intensity back to a stimulation value that was previously determined to be acceptable.

If successive increases to the stimulation intensity do not cause a definitive improvement in sleep quality (i.e. if an SQM computed for the most recent does not indicate improved sleep quality when compared to one or more historical SQMs) then the stimulation intensity will be decreased (e.g. in step 309) to the lowest known intensity that was found to result in an equivalent SQM. In doing so, system 100 can conserve energy by preventing the delivery of unnecessarily high stimulation when a lower intensity stimulation could be used just as effectively, thereby improving power efficiency compared to some conventional OSA stimulators.

In various examples, subprocess 311 might continue to iterate for a predetermined number of cycles, until the algorithm determines that a target parameter has been sufficiently optimized (i.e. when sleep quality improvements cease to occur), until a patient input is received suggesting that an adjustment caused discomfort, or until controller 102 receives a command from external controller 105 to stop the titration process.

Although algorithm 300 provides a simplified version of a process for titrating stimulation that is applied during one particular sleep stage, those skilled in the art will appreciate that various modifications could be made to, e.g., simultaneously titrate a plurality of parameters for a particular sleep stage, or to titrate one or more parameters for each sleep stage of a plurality of sleep stages simultaneously. For instance, a processor could be configured to run multiple separate instances of algorithm 300 in tandem, to simultaneously titrate stimulation parameters for each sleep stage. Accordingly, different examples of titration 300 might include more or fewer steps and should not be misinterpreted as being strictly limited to only the illustrated example.

FIGS. 4A-4C are examples of prompts 402 which might be transmitted to a patient during steps 308 or 320 of the titration 300 shown in FIG. 3. These urge the patient to provide an input 404 assessing whether they experienced any discomfort due to therapy. Prompts 404 are ideally presented to the patient in the morning following a therapy adjustment. However, these could be presented to the patient at regular intervals or whenever the controller 102 detects that sleep quality has deviated from the norm. These could be quantitative or qualitative in various embodiments.

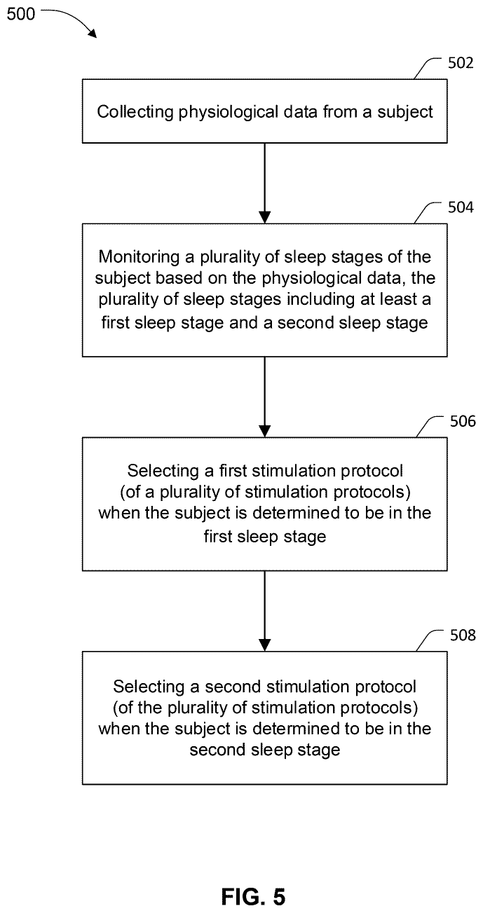

With reference to FIG. 5, a flowchart is provided in accordance with a method 500 for selecting a stimulation protocol based on the sleep stage of a subject. In particular, method 500 includes collecting 502 physiological data and monitoring 504 a plurality of sleep stages experienced by a subject. The plurality of sleep stages includes at least a first sleep stage (e.g. light sleep) and a second sleep stage (e.g. deep sleep). In certain examples, the plurality of sleep stages might correspond with some or all of the sleep stages selected from the following list: light sleep (N1), intermediate sleep (N2), deep sleep (N3), and REM sleep. Method 500 continues such that, when the subject is determined 506 to be in the first sleep stage, controller 102 selects the first stimulation protocol and optionally instructs the IPG 101 to apply stimulation a nerve (e.g. the HGN) using the first stimulation protocol. Further, when the subject is determined to be in the second sleep stage, controller 102 selects 508 the second stimulation protocol and optionally instructs the IPG 101 to apply stimulation. In various examples, a given stimulation protocol might be selected and not applied if, for instance, the system 100 is configured to provide closed-loop stimulation, such that stimulation is only applied in response to detecting a physiological trigger/event (e.g. stimulation might only be applied in response to detecting an apnea and/or a hypopnea), and no such event was detected before the patient transitioned into another sleep stage.

With reference to FIG. 6, method 600 provides steps in accordance with a titration for incrementally adjusting the value of one or more parameters of a stimulation protocol that is selectively applied during a particular sleep stage. The method 600 comprises: collecting 602 physiological data over at least a first period and a second period; detecting 604 respiratory event(s), based on the physiological data; determining 606 when the subject is in one sleep stage (of a plurality of sleep stages) based on the physiological data; triggering 608 an IPG to generate electrical pulses using a first stimulation protocol (of a plurality of stimulation protocols) at a first stimulation intensity, in response to each respiratory event detected in the one sleep stage during the first period; generating 610 a first SQM, based on a quantity of respiratory event(s) detected during the one sleep stage using the physiological data collected over the first period; receiving 612 a first patient input; adjusting 614 the first stimulation protocol to a second stimulation intensity, based on the patient input and/or based on the physiological data; triggering 616, (during the second period) the IPG to generate electrical pulses using the first stimulation protocol at the second stimulation intensity each time the respiratory event is detected in the one sleep stage; generating 618 a second sleep quality metric (“SQM”) based on a quantity of respiratory event(s) detected during the one sleep stage, using the physiological data collected over the second period; receiving 620 a second patient input; comparing 622 the first SQM to the second SQM; determining 624 if the first stimulation protocol should be further adjusted, based on the comparison and/or based on the second patient input; and similarly titrating 626 each stimulation protocol of the plurality of stimulation protocols for a respective sleep stage of the plurality of sleep stages, to identify an optimal stimulation intensity for each sleep stage of the plurality of sleep stages.

In alternate embodiments, either method 500 or 600 could include more or fewer steps, and additional steps might relate to any of the aspects, features, or components discussed throughout the body of this disclosure.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. In the above discussion, processes, elements, and techniques that are not necessary for those with ordinary skill in the art to gain a complete understanding of the invention may not be described. The entire disclosure of each patent and publication cited herein is incorporated by reference, as if each such patent or publication were individually incorporated by reference herein. Various features and advantages of the invention are set forth in the following claims.

What is claimed is:

1. A method for adjusting a stimulation protocol configured for use during one sleep stage of a plurality of sleep stages, the method comprising:
collecting, via at least one sensor, physiological data from a subject over at least first and second periods, the second period being after the first period;
detecting respiratory events, based on the physiological data;
determining when the subject is in the one sleep stage, based on the physiological data;
generating, for the one sleep stage, a first sleep quality metric (“SQM”), using physiological data collected over the first period;
generating, for the one sleep stage, a second SQM, using physiological data collected over the second period;
receiving a patient input indicative of a comfort level;
comparing the first SQM to the second SQM; and
determining, based on the comparison and/or the patient input, if the stimulation protocol should be adjusted to either increase or decrease stimulation intensity.

2. The method of claim 1, wherein the plurality of sleep stages includes at least: light sleep, deep sleep, and REM sleep.

3. The method of claim 2, wherein the method is conducted for each sleep stage of the plurality of sleep stages, such that the stimulation intensity is independently adjusted for each sleep stage of the plurality of sleep stages.

4. The method of claim 1, wherein adjusting the stimulation intensity comprises adjusting a value of a parameter of the stimulation protocol.

5. The method of claim 4, wherein the parameter comprises at least one of: a pulse amplitude, a pulse width, a pulse frequency, and/or a duty cycle.

6. The method of claim 1, further comprising: classifying the detected respiratory events as being at least one of an apnea event and/or a hypopnea event.

7. The method of claim 6, wherein generating the first SQM and the second SQM includes determining that, during the first and second periods, respectively, first and second quantities of apneas and/or hypopneas were detected while the subject was in the one sleep stage, wherein a higher SQM is indicative of decreased sleep quality.

8. The method of claim 7, further comprising: increasing the stimulation intensity, based on the determination that the second SQM is less than the first SQM.

9. The method of claim 7, further comprising: decreasing the stimulation intensity, based on the determination that the second SQM is greater than or equal to the first SQM.

10. The method of claim 7, further comprising: decreasing the stimulation intensity, when the patient input indicates discomfort.

11. The method of claim 10, wherein the stimulation intensity is decreased by a predetermined percentage based on the patient input.

12. The method of claim 1 including determining, for each of the plurality of sleep stages, a maximum value of the stimulation intensity corresponding with a therapeutic threshold that lies below a pain threshold of the subject, based on the patient input, and based on the comparison of first and second SQMs.

13. The method of claim 1, wherein the at least one sensor comprises at least one of: an inertial measurement unit (IMU), a pressure sensor, an accelerometer, a sound sensor, a gyroscope, a heart rate monitor, an electrocardiogram (ECG) sensor, a blood pressure sensor, a blood oxygen level sensor, an electromyography (EMG) sensor, and/or an electroneurogram (ENG) sensor.

14. The method of claim 1, wherein the patient input is received as a digital input on an external remote.

15. The method of claim 1 further including, applying the stimulation protocol with an implantable pulse generator.

16. A method for selecting one of a plurality of stimulation protocols, the method comprising:
collecting physiological data from a subject, using at least one sensor;
monitoring a plurality of sleep stages of the subject based on the physiological data, the plurality of sleep stages including at least a first sleep stage and a second sleep stage;
selecting a first stimulation protocol of the plurality of stimulation protocols when the subject is determined to be in the first sleep stage; and
selecting a second stimulation protocol of the plurality of stimulation protocols when the subject is determined to be in the second sleep stage.

17. The method of claim 16, wherein the first stimulation protocol and the second stimulation protocol differ with respect to a value of at least one parameter.

18. The method of claim 17, wherein the at least one parameter comprises: a pulse amplitude, a pulse width, a pulse frequency, and/or a duty cycle.

19. The method of claim 17, wherein the plurality of sleep stages includes light sleep, deep sleep, and REM sleep.

20. The method of claim 17, further comprising: detecting at least one respiratory event, based on the physiological data, the at least one respiratory comprising at least one of: an inspiratory event, an apnea event, and/or a hypopnea event.

21. The method of claim 20, further comprising: applying electrical stimulation to a nerve in response to detecting the respiratory event.

22. The method of claim 20, further comprising: performing a titration to optimize each one of the plurality of stimulation protocols for a respective sleep stage of the plurality of sleep stages.

23. The method of claim 16, wherein optimizing each one of the plurality of stimulation protocols comprises determining, for the respective sleep stage, a maximum value of a stimulation intensity corresponding with a therapeutic threshold that lies below a pain threshold, based on the patient input.

24. The method of claim 16, wherein the at least one sensor comprises at least one of: an inertial measurement unit (IMU), a pressure sensor, an accelerometer, a sound sensor, a gyroscope, a heart rate monitor, an electrocardiogram (ECG) sensor, a blood pressure sensor, a blood oxygen level sensor, an electromyography (EMG) sensor, and/or an electroneurography (ENG) sensor.

25. A stimulation system configured for selecting a stimulation protocol from a plurality of stimulation protocols, in response to detecting a transition in the sleep stage of a subject, the plurality of stimulation protocols including at least a first stimulation protocol and a second stimulation protocol, the system comprising:
at least one sensor configured for collecting physiological data; and
a controller communicatively linked to the at least one sensor, the controller being configured to process the physiological data to:
monitor a plurality of sleep stages, the plurality of sleep stages including at least a first sleep stage and a second sleep stage;
determine when the subject is in the first sleep stage; and
determine when the subject transitions from the first sleep stage to the second sleep stage;
select the first stimulation protocol when the subject is in the first sleep stage; and
select the second stimulation protocol when the subject transitions to the second sleep stage.

26. The system of claim 25, wherein the plurality of sleep stages includes: light sleep, deep sleep, and REM sleep.

27. The system of claim 25, wherein the at least one sensor comprises one or more of: an inertial measurement unit (IMU), a pressure sensor, an accelerometer, a sound sensor, a gyroscope, a heart rate monitor, an electrocardiogram (ECG) sensor, a blood pressure sensor, a blood oxygen level sensor, an electromyography (EMG) sensor, and/or an electroneurography (ENG) sensor.

28. The system of claim 27, wherein the stimulation system comprises an implantable pulse generator (“IPG”) electrically coupled to a nerve.

29. The system of claim 28, wherein the at least one sensor includes an implantable sensor that is housed within the IPG.

30. The system of claim 29, wherein the at least one sensor includes an external sensor that is worn by the subject.

31. The system of claim 25, wherein the controller is configured to process the physiologic data to detect a respiratory event comprising at least one of: an inspiratory phase, an apnea, and/or a hypopnea.

32. The system if claim 31, wherein, the IPG is configured to transmit electrical stimulation to the nerve using the selected stimulation profile in response to detecting the respiratory event.

33. The system of claim 32, including a patient remote in wireless communication with the IPG and configured to receive a patient input.

34. The system of claim 33, wherein the patient input is indicative of a perceived comfort level following stimulation.

35. The system of claim 34, wherein the controller is configured to adjust a stimulation intensity corresponding to at least one of the plurality of stimulation protocols, based on the patient input.

36. The system of claim 25, wherein the first stimulation protocol and the second stimulation protocol differ with respect to a value of at least of: a pulse amplitude, a pulse width, a pulse frequency, and/or a duty cycle.
